(19) United States
(12) Patent Application Publication
(10) Pub. No.: US 2025/0177675 A1
(43) Pub. Date: Jun. 5, 2025

(54) Device to Securely and Dependably Hold Nasal Prongs in Place without Irritation or Constricting the Cannula Tube and Method of Using

(71) Applicants: Steven Lee Sugarek, Houston, TX (US); Chester Alan Sugarek, Freeport, TX (US)

(72) Inventors: Steven Lee Sugarek, Houston, TX (US); Chester Alan Sugarek, Freeport, TX (US)

(21) Appl. No.: 19/050,656

(22) Filed: Feb. 11, 2025

(51) Int. Cl. A61M 16/06

(52) U.S. Cl. CPC A61M 16/0666; A61M 2205/0216; A61M 2205/025; A61M 2209/088; A61M 2210/0618

(57) ABSTRACT

Disclosed is an elastomeric device and method of using wherein the device is fitted to cannula tubing to securely and dependably hold nasal prongs comfortably in a user's nose. The device comprises a flange and body with a cut though the body. The device is positioned with flange located behind the ear facing the front and contacting the head. The inside diameter of the device nearest the flange is slightly larger than the outside diameter of the cannula tube to minimize constricting the tube. The device's position is adjusted by the wearer to a point where the flange contacts the upper back of the wearer's ear and side of the head. The device holds the nasal prongs securely and dependably in a preferred position with enough tension to keep the nasal prongs in place without irritating the user, constricting the cannula tubing, or requiring a taut bolo slide.

### CROSS-REFERENCES RELATED APPLICATIONS

Not applicable.

### STATEMENT REGARDING FEDERALLY SPONSORED RESEARCH OR DEVELOPMENT

Not applicable.

### NAMES OF THE PARTIES TO A JOINT RESEARCH AGREEMENT

Not applicable.

### REFERENCE TO A “SEQUENCE LISTING”

Not applicable.

### STATEMENT REGARDING PRIOR DISCLOSURES BY THE INVENTOR OR A JOINT INVENTOR

Not applicable.

### BACKGROUND OF THE INVENTION

### 1. Field of the Invention

This invention relates to a device to securely and dependably hold in place nasal prongs in the nasal cavities of a human using frictional contact between the device and a wearer's skin behind the ears without irritating the user, constricting the cannula tubing, or requiring a taut bolo slide.

This invention further relates to a method of using the device to hold nasal prongs in the nasal cavities of a human.

### 2. The Current State of the Art

Certain health conditions, especially respiratory ailments, make it hard for a user to receive enough oxygen. In these cases, receiving supplemental oxygen through a nasal cannula may be necessary.

A nasal cannula provides a user supplemental oxygen or oxygen therapy through the nose. Nasal cannula is more comfortable and practical than a face mask and is best for long-term use. The oxygen is supplied by a thin, flexible tube that goes around the user's ears and into the nose. Nasal prongs are two prongs that go inside the nostrils to deliver the oxygen. The tube is connected to an oxygen supply.

The amount of supplemental oxygen depends on the user's need. The use of a nasal cannula begins with selecting the correct tubing diameter based on the required oxygen flow rate; typically, 2 mm to 6 mm outside diameter. The nasal prongs are selected to be long enough to enter the nostrils fully but not so large to occlude them. The cannula tubing is then connected to the oxygen source and set at the desired flow rate.

The most common technique for installing nasal cannula starts with placing the assembly in front of the chest and inserting the nasal prongs in the nostrils. After the nasal prongs are in place the tubing is directed from the nasal prongs to over the ears from front to back. The tubes are then brought down behind the ears and underneath the chin to the front of the chest. If the tubes have a bolo slide, they are then secured under the chin by moving the bolo slide so the tubing is snug but not too tight.

The principle technical challenge with nasal cannula and appliances or structures to support the tubes is that the nasal prongs are still subject to fall out of the nose. Solutions to this technical challenge have heretofore been various head and neck straps, retrofittable flexible ear pieces, and inflexible structures connected to the cannula tubes to help hold the nasal prongs in place. None of these retrofitted appliances securely and dependably hold the nasal prongs in the nose and without constricting the tubes.

The head and neck straps and inflexible structures are known to irritate the user, particularly when confined to a bed or sleeping. The slit flexible ear pieces are known to become dislodged from the tubes causing the nasal prongs to fall out of the nose. As a work-around, users tightly secure the cannula tubes by a bolo slide at the front of the chest to hold the nasal prongs in place. The required tightness caused by the bolo slide frequently irritates the ears or crimps the tubes resulting in obstruction of oxygen flow. The bolo slide may not remain in place again allowing the nasal prongs to fall out of the nose.

The invention disclosed here overcomes these technical challenges. The disclosed device is an appliance made of a soft, pliable, and moldable elastomer attached to the cannula tubes at the point of manufacture. It securely and dependably holds the nasal prongs in place via friction between the head and ears without constricting the tubes, irritating the user, or requiring a taut bolo slide.

### 3. Description of the Related Art Including Information Disclosed Under 37 C.F.R. 1.97 and 1.98

There are numerous patents and published patent application in the field of the invention disclosed here for keeping nasal prongs in place, but none anticipate nor render the invention obvious. No U.S. patents or published U.S. patent applications not otherwise issuing as a patent are relevant here as prior art in the field of devices that hold in place nasal prongs using frictional contact between the device and the tube and a wearer's skin without irritating the user, constricting the tubing, or requiring a taut bolo slide.

Some prior art restraints for nasal prongs are straps connected to the tubing and passing over and/or behind the head. They relieve some of the weight of the cannula tube that would normally press on the ears or work to hold the cannula tubes in place, and, in turn, the nasal prongs. They are U.S. Patent Appl. No. US 2007/0056590, Wolfson, I.A., Holder for Nasal Cannula; U.S. Patent Appl. No. US 2011/0203595, Hashemieh, J., Cannula Security Piece; U.S. Patent Appl. No. US 2013/0146064, Dryden, H., Nasal Cannula Support Device; U.S. Patent Appl. No. US 2023/0277795, Kim, J., et al., Headgear Assembly for Securing a Fluid Tube; U.S. Patent Appl. No. US 2024/0261527, Metelits, J.B., et al., Nasal Cannula; U.S. Patent Appl. No. US 2024/0366901, U.S. Pat. No. 4,836,200, Clark, R.D. and Egan, S.D., Oxygen Tube Support Strap; U.S. Pat. No. 5,438,979, Johnson, Jr., A.L. and Stenger, T.K., Nasal Cannula Support; U.S. Pat. No. 6,536,436, McGlothen, R., Strap for Nasal Cannula; U.S. Pat. No. 7,735,490, Rinaldi, T.L., Adjustable Nasal Cannula Apparatus and Method of Use; U.S. Pat. No. 8,701,669, Jackman, R.L. and Anderson, K.J., Nasal Cannula Positioning Device.

Others rely on covering the cannula tubes in a retrofitted slit soft or compressible material which passes over the ears. The slit permits these devices to be applied at the point of use. They are U.S. Patent Appl. No. US 2022/0007752, Sason, R., Ear Protector Hook; U.S. Patent Appl. No. 2024/0366901, Sugarek, S.L. and Sugarek, C.A., Device to Hold a Nasal Cannula in Place without Irritation or Compressing the Cannula Tube and Method of Using; U.S. Pat. No. 4,699,139, Marshall, M.F. and Kislow, N.C., Nasal Cannula Assembly with Patient Comfort Pad; U.S. Pat. No. 4,949,733, Sampson, R.D., Nasal Oxygen Cannula Pad; U.S. Pat. No. 5,025,805, Nutter, B., Nasal Cannula Assembly; U.S. Pat. No. 6,986,353, Wright, C.A., Divided Nasal Cannula Assembly.

Still others rely on structures and/or clips, nasal cups, nasal inserts, or facial adhesives to hold the nasal prongs in place and support the cannula tubes to relieve pressure on the ears. All are subject to fail or constrict the cannula tube. They are U.S. Pat. No. 4,106,505, Salter, P.W. and Cook, W.W., Nasal Cannula Assembly; U.S. Pat. No. 4,216,769, Grimes, J.L., Bi-Flow Nasal Cup; U.S. Pat. No. 4,753,233, Grimes, J.L., Nasal Cannula; U.S. Pat. No. 6,669,712, Cardoso, N., Nasal Oxygen Cannula with Supply Tube Management; U.S. Pat. No. 10,261,339, Sugarek, S., Skin Contact Reduction Device Eyewear and Other Human Ear and Temple Resting Equipment; U.S. Pat. No. 11,491,295, Quechuleno, C., Nasal Cannula Clip System and Method.

### BRIEF SUMMARY OF THE INVENTION

Other than positioning the tubes around the ears, nasal cannula in use today is fitted without a positioner of any kind. The nasal prongs are primarily held in position by tensile force created by draping the tubes over the ears creating frictional contact between the ears and head, pulling the tubes tight in front of the chest, and securing them with a bolo slide. Nevertheless, the nasal prongs tend to fall out of position when the head is turned side-to-side or up-and-down. It is well known that individuals under hospital care are often bed-ridden and may be left alone for extended periods of time. At these times, if the nasal prongs fall out of the nose, the loss of supplemental oxygen can create serious health problems for the user, and even death.

Many cannula skin contact reduction devices in current use are installed on the cannula tube at the point of use. They typically are cut or slit longitudinally to permit the device to fit over the tube. The cut or slit makes the device prone to falling off the tube. Damage can also occur to the tube because skin contact reduction devices may constrict the tubing.

To keep the nasal prongs inserted in a user's nostrils securely and dependably, the device disclosed here is fitted to the cannula tubes at the point of manufacture. The dimensions of the device disclosed here and its embodiments are gauged to the outside diameter of the tube to which it is to be fitted, thereby being useful for a wide range of tubing sizes.

The device comprises a flange connected indistinguishably to a body. The flange blade is patent; that is, it is not cut radially from the inside diameter to the outside. The patent flange blade ensures that once the device is placed over a cannula tube it will not become dislodged from the tube. The body of the device is sliced through longitudinally in various patterns. The cut in the body slightly reduces the structural integrity of the device when it is positioned around the tube. The reduction in structural integrity permits the device to deform slightly when in use minimizing constricting the tube and skin irritation.

When loading is applied to the flange, it flexes back along the body applying a small amount of pressure on the tubing, therefore eliminating slippage. This effect combined with the natural friction that is found when the device contacts the tubing ensures fit without slippage. It further minimizes or eliminates the need for a tightly fitted bolo slide.

These features with other technological improvements, which will become subsequently apparent, reside in the details as more fully described hereafter and claimed, reference being had to the accompanying drawings forming a part hereof.

### BRIEF DESCRIPTION OF THE SEVERAL VIEWS OF THE DRAWINGS

The present application is more fully understood by reference to the following figures, which are for illustrative purposes only. The figures are not necessarily drawn to scale and elements of similar structures or functions are represented by like reference numerals for illustrative purposes throughout the figures. The figures are only intended to facilitate the description of the various embodiments described herein. The figures do not describe every aspect of the teachings disclosed herein and do not limit the scope of the claims.

FIG. 1 illustrates the preferred embodiment of the device.

FIG. 2 shows the preferred embodiment of the device in cross-section.

FIG. 3 depicts the preferred embodiment in place around a cannula tube.

FIG. 4 pictures a second embodiment.

FIG. 5 illustrates a third embodiment.

FIG. 6 shows the preferred embodiment in place on the cannula tube prior to placing it on a user.

FIG. 7 depicts the preferred embodiment being positioned behind a user's ear.

FIG. 8 pictures the preferred embodiment in final position behind the ear.

### DETAILED DESCRIPTION OF THE INVENTION

### 1. Glossary of Terms Used in the Disclosure

The phrase “nasal cannula” refers to the entire device used to deliver oxygen through the nose. It includes tubing, nasal prongs, bolo slide, and parallel wye connector.

The phrase “nasal prongs” refer to the assembly containing the two prongs that are inserted in the nostrils which is a part of the nasal cannula.

The words “tube” or “tubes” and phrases “cannula tube”, “cannula tubes,” or “cannula tubing” refer to the two flexible tubes with each of their ends connected to the nasal prongs. The opposite ends of the tubes are further connected to a parallel wye connector known to a person having ordinary skill in the art which in turn connects to an oxygen supply.

### 2. List of Reference Numbers

### Number Description

100 Preferred embodiment
102 Body
104 Flange
106 Hole in the body
108 Spiral cut
110 Hole in the flange that may extend into the body
112 A circle circumscribed by a point at which the hole in the flange meets the hole in the body
300 Cannula tube
302 Space between cannula tube and flange
400 Second embodiment
402 Longitudinal cut along the length of the body
500 Third embodiment
502 Cut partially encircling the body and extending from the bottom of the body to midway along the length of the body
504 Point midway from the bottom of the body to a point at which the body meet the flange
506 Cut partially encircling the body and extending from midway along the length of the body to the point at which the cut meets the flange
600 Oxygen supply tube
602 Parallel wye connector
604 Second tube
606 Bolo slide
608 Nasal prongs
700 User
702 User's ear

### 3. Detailed Descriptions of the Preferred and Other Embodiments of the Device

The preferred and other embodiments require that they have the physical structures as disclosed here and their dimensions determined by the outside diameter of cannula tube 300, depicted in FIG. 3, to which they are attached.

The soft, pliable, and moldable elastomer is a medical grade silicone-based polymer or any other polymer suitable for non-implantable body contact and having a Shore A hardness ranging from 20 to 80, preferably Shore A 20 to 30.

### (a) Detailed Description of the Preferred Embodiment

FIG. 1 shows the preferred embodiment 100. It comprises a body 102 and flange 104. 102 and 104 are made of the same soft, pliable, and moldable elastomer. 102 and 104 are indistinguishably connected to form one piece with coinciding centerlines. The center of hole 110 is located nominally at the centerline of 104 and passes straight through 104. The center of hole 106 is located nominally at the centerline of 102 and extends uniformly upward from the bottom of 102 to 112, the place where 106 first contacts hole 110.

The outside diameter of flange 104 is nominally 4 to 12 times the outside diameter of 300, preferably 6 times.

The outside diameter of body 102 is always less than the same of flange 104 and otherwise nominally 2 to 6 times the outside diameter of the 300, preferably 3 times.

The inside diameter of hole 110 in flange 104 is nominally 1.2 to 3 times the outside diameter of 300, preferably 2 times.

The inside diameter of hole 106 in body 102 is nominally 0.8 to 1.2 times the outside diameter of 300; preferably equal.

The length of body 102 as measured from the end opposite flange 104 to the point at which it first contacts 104 is nominally 1 to 10 times the outside diameter of 300, preferably 5 times.

The thickness of flange 104 as measured from top to bottom, is nominally 0.2 to 4 times the outside diameter of 300, preferably 0.75 times.

The depth of hole 110 as measured from the top of flange 104 to 112 ranges nominally from equal to the thickness of 104 to 0.75 times the sum of the thickness of flange 104 and the length of body 102, preferably 0.2 times the sum of the thickness of flange 104 and the length of body 102.

FIG. 1 also shows spiral cut 108 made through the side of 102 from outside to inside nominally tracking 180 to 720 degrees from the end of body 102 opposite 104 to the point where 102 first contacts 104, preferably 360 degrees. When viewed from flange 104, FIG. 1 depicts spiral cut 108 tracking clockwise from the end of body 102 opposite 104 to the point where 102 first contacts 104. Persons with ordinary skill in the art understand that spiral cut 108 can track counterclockwise and still be within the scope of the disclosure.

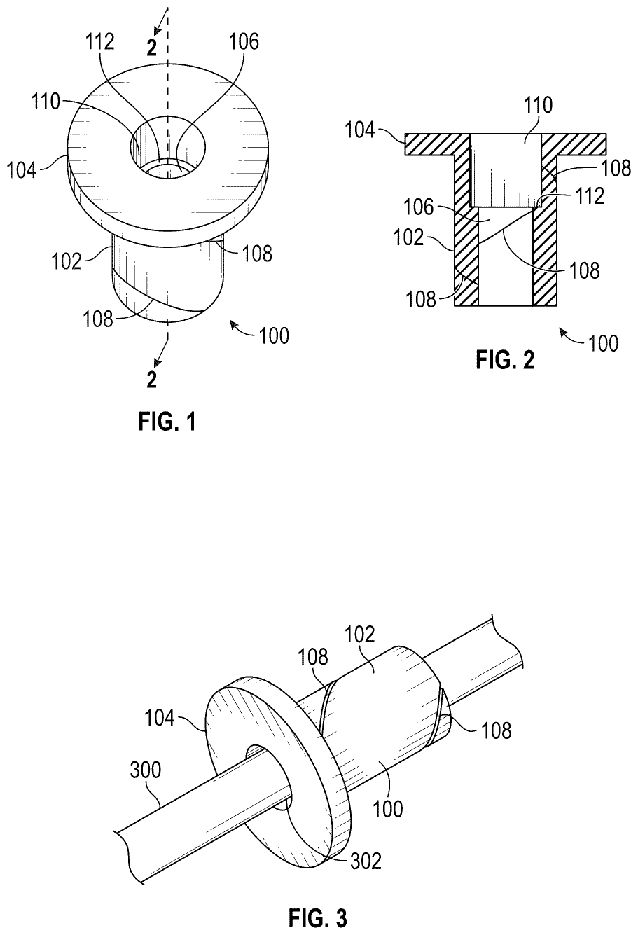

FIG. 2 is a cross-sectional view of 100. It depicts in greater detail that in the preferred embodiment the inside diameter of hole 110 is slightly larger than the same of 106. It also shows 106 extending from the bottom of 102 to the point where hole 106 meets hole 110 at 112. 110 extends from 112 through to the top of 104.

FIG. 3 shows 100 in place around tube 300. It further shows that when 100 is placed around 300, a space 302 between the cannula tube and flange is always maintained between the outside of the cannula tube and inside of hole 110. Space 302 minimizes the likelihood that 100 will constrict tube 300 when in use.

FIG. 3 also shows 108, a spiral cut encircling body 102. The spiral cut permits 100 to slightly flex while in use. The flexing allows 100 to maintain its initial position behind the ear while again minimizing the likelihood that 100 will constrict tube 300 when in use, or irritate the user.

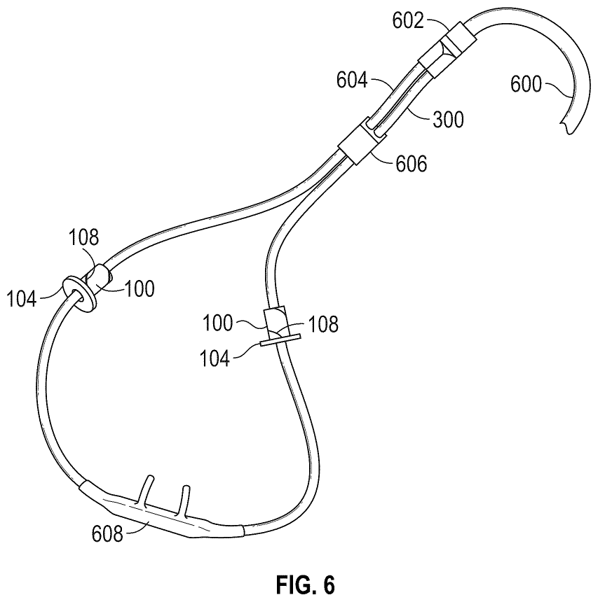

FIG. 6 depicts a nasal cannula ready for use. One each of items 100 are slid over tube 300 and tube 604 at the point of manufacture with flange 104 of each of 100 positioned to face nasal prongs 608. Parallel wye connector 602 and bolo slide 606 are typically installed at the point of manufacture. FIG. 6 also shows oxygen supply tube 600 connected to the upstream side of 602. 600 is typically fitted to 602 at the point of use.

### (b) Detailed Descriptions of the Other Embodiments

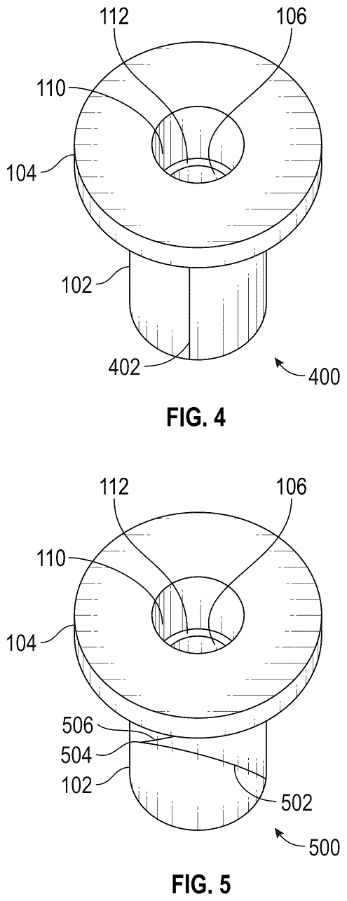

Two additional embodiments are disclosed and depicted in FIGS. 4 and 5. Many of the structures and items depicted in 100 are duplicated in embodiments 400 and 500.

Items 102, 104, 106, 110, and 112 are duplicated in FIGS. 4 and 5.

FIG. 4 shows second embodiment 400. FIG. 4 shows longitudinal cut 402 made through the side of body 102 coursing straight from the end of 102 opposite 104 to the point where 102 meets 104.

FIG. 5 shows third embodiment 500. FIG. 5 shows cut 502 made through the side of 102 nominally tracking 90 to 270 degrees, preferably 180 degrees, from right-to-left from the bottom of 102 to the point where 502 first contacts 504, the nominal longitudinal midpoint of 102. From point 504, cut 506 is made through the side of 102 nominally tracking 90 to 270 degrees, preferably 180 degrees, from left-to-right from 504 to the point where 506 first contacts 104. Persons with ordinary skill in the art understand that cut 502 can track from left-to-right and cut 506 can track from right-to-left, meeting at point 504, and still be within the scope of the disclosure.

### 4. Detailed Description of the Method of Using the Preferred and Other Embodiments of the Device

FIG. 6 depicts the first step in using devices 100, 400, or 500. If not already fitted at the manufacturer, oxygen supply tube 600 is fitted to the upstream end of parallel wye connector 602. Tubes 300 and 604 are typically connected to the downstream end of 602 at the point of manufacture.

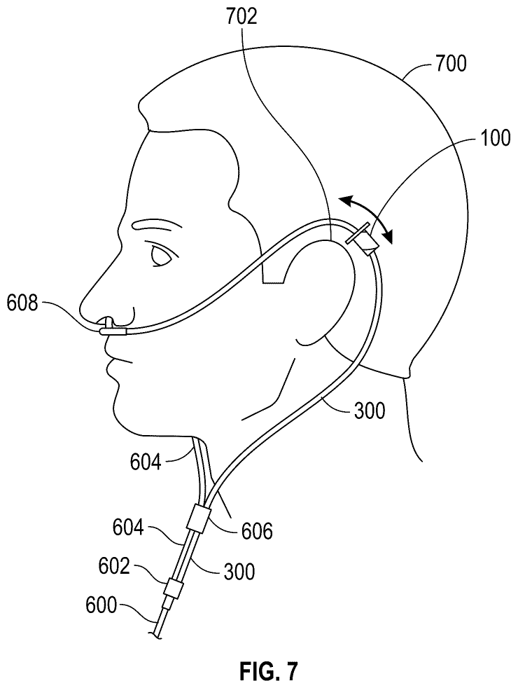

Persons with ordinary skill in the art know the most common technique for installing nasal cannula starts with placing the assembly in front of the chest and comfortably inserting the nasal prongs in the nostrils. After the nasal prongs are in place the tubing is directed from the nasal prongs to over the ears from front to back. FIG. 7 illustrates this by showing the initial positioning of tube 300 with 100 provisionally fitted to it behind ear 702. Another 100 fitted to 604, but not shown, but understood by persons with ordinary skill in the art, is positioned loosely behind the other ear. A 400 or 500 may also be provisionally positioned on 300, and another on 604, not shown, but understood by persons with ordinary skill in the art, so each fits loosely behind the ear.

300 and 604 are then pulled towards the back of the head through each of 100, 400, or 500 until snug behind the ears and 608 is securely and dependably held in place. The remaining of 300 and 604 passing behind the ears are then laid on the chest if user 700 is laying down or hung loosely in front of the chest if standing.

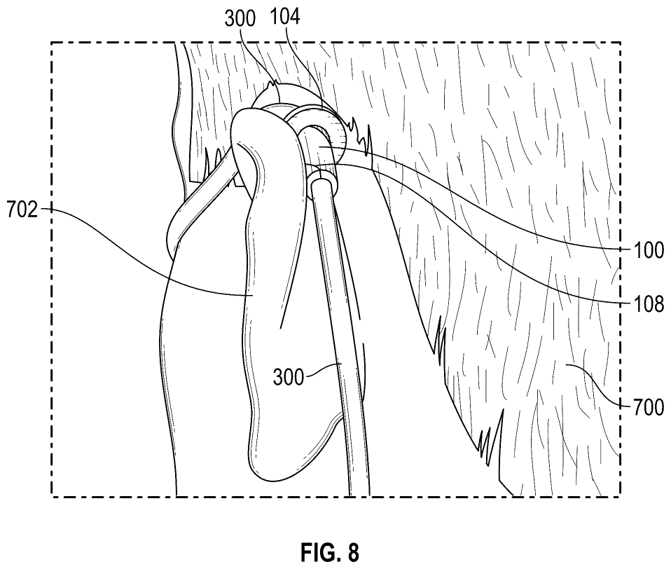

FIG. 8 shows the final position of a 100, 400, or 500 on tube 300. 300 rests on the skin between ear 702 and the side of the head of 700. At this point 300 and 604, not shown, are at their proper positions, and 100, 400, or 500 has been slid over 300 to a point snug behind ear 702. If necessary, bolo slide 606 may be cinched comfortably under the chin of 700.

Persons having ordinary skill in the art of dependably securing nasal prongs in the nasal cavities while also preventing irritation of the skin will understand that the device and method of using it described in the preferred and other embodiments can vary and remain within the invention herein described. Variations obvious to those persons with ordinary skill in the art are included in the invention.

This written description uses examples to disclose the invention, including the preferred embodiment, and to enable a person having ordinary skill in the relevant art to practice the invention, including making and using any of the devices disclosed here or and performing any incorporated methods. The patentable scope of the invention is defined by the claims, and may include other examples that occur to those persons of ordinary skill in the art. Such other examples are intended to be within the scope of the claims if they have structural elements that do not differ from the literal language of the claims, or if they include equivalent structural elements with insubstantial differences from the literal language of the claims.

Further, multiple variations and modifications are possible in the embodiments of the invention described here. Although certain illustrative embodiments of the invention have been shown and described here, a wide range of modifications, changes, and substitutions is contemplated in the foregoing disclosure. In some instances, some features of the present invention may be employed without a corresponding use of the other features. Accordingly, it is appropriate that the foregoing description be construed broadly and understood as being given by way of illustration and example only, the spirit and scope of the invention being limited only by the appended claims.

We claim:

1. A device made of a soft, pliable, and moldable elastomer to securely and dependably hold nasal prongs in a nasal cavity of a human by attachment to a cannula tube, said cannula tube having a first outside diameter, comprising;
a. a flange comprising a first cylinder having a thickness and a second outside diameter;
(1) said thickness less than said second outside diameter;
b. a body comprising a second solid cylinder having a length and a third outside diameter;
(1) said length greater than said third outside diameter;
c. the second outside diameter is greater than the third outside diameter;
d. said flange having a first longitudinal centerline;
e. said body having a second longitudinal centerline;
f. the flange indistinguishably and permanently joined to one end of the body so that said first longitudinal centerline and said second longitudinal centerline are coincident;
g. a first hole passing through the flange with centerline equal to the first longitudinal centerline;
(1) said first hole having a first inside diameter less than the third outside diameter;
h. a second hole passing through the body with centerline equal to the second longitudinal centerline;
(2) said second hole having a second inside diameter less than the third outside diameter and less than or equal to said first inside diameter;
i. depth of the first hole as measured from top of the flange downward into the body is less than or equal to a sum of the thickness and the length; and
j. a cut through side of the body.

2. The device in claim 1 wherein;
a. said second outside diameter of said flange is 4 to 12 times said first outside diameter;
b. said third outside diameter of said body is 2 to 6 times the first outside diameter;
c. said thickness of the flange is 0.2 to 4 times the first outside diameter;
d. said length of the body is 1 to 10 times the first outside diameter;
e. said first inside diameter of said first hole is 1.2 to 3 times the first outside diameter;
f. said second inside diameter of said second hole is 0.8 to 1.2 times the first outside diameter;
g. said depth of the first hole ranges from equal to the thickness to 0.75 times said sum of the thickness and the length; and
h. said cut courses spirally 180 to 720 degrees from an end of the body farthest from the flange to the flange.

3. The device in claim 1 wherein;
a. said second outside diameter of said flange is 6 times said first outside diameter;
b. said third outside diameter of said body is 3 times the first outside diameter;
c. said thickness of the flange is 0.75 times the first outside diameter;
d. said length of the body is 5 times the first outside diameter;
e. said first inside diameter of said first hole is 2 times the first outside diameter;
f. said second inside diameter of said second hole is equal to the first outside diameter;
g. said depth of the first hole is 0.2 times said sum of the thickness and the length; and
h. said cut courses spirally 360 degrees from an end of the body farthest from the flange to the flange.

4. The device in claim 1 wherein;
a. said second outside diameter of said flange is 4 to 12 times said first outside diameter;
b. said third outside diameter of said body is 2 to 6 times the first outside diameter;
c. said thickness of the flange is 0.2 to 4 times the first outside diameter;
d. said length of the body is 1 to 10 times the first outside diameter;
e. said first inside diameter of said first hole is 1.2 to 3 times the first outside diameter;
f. said second inside diameter of said second hole is 0.8 to 1.2 times the first outside diameter;
g. said depth of the first hole ranges from equal to the thickness to 0.75 times said sum of the thickness and the length; and
h. when viewed from the flange, said cut courses spirally clockwise or counterclockwise 90 to 270 degrees from an end of the body farthest from the flange to a longitudinal midpoint of the body and then spirally in an opposite direction 90 to 270 degrees from said longitudinal midpoint to the flange.

5. The device in claim 1 wherein;
a. said second outside diameter of said flange is 6 times said first outside diameter;
b. said third outside diameter of said body is 3 times the first outside diameter;
c. said thickness of the flange is 0.75 times the first outside diameter;
d. said length of the body is 5 times the first outside diameter;
e. said first inside diameter of said first hole is 2 times the first outside diameter;
f. said second inside diameter of said second hole is equal to the first outside diameter;
g. said depth of the first hole is 0.2 times said sum of the thickness and the length; and
h. when viewed from the flange, said cut courses spirally clockwise or counterclockwise 180 degrees from an end of the body farthest from the flange to a longitudinal midpoint of the body and then spirally in an opposite direction 180 degrees from said longitudinal midpoint to the flange.

6. The device in claim 1 wherein;
a. said second outside diameter of said flange is 4 to 12 times said first outside diameter;
b. said third outside diameter of said body is 2 to 6 times the first outside diameter;
c. said thickness of the flange is 0.2 to 4 times the first outside diameter;
d. said length of the body is 1 to 10 times the first outside diameter;
e. said first inside diameter of said first hole is 1.2 to 3 times the first outside diameter;
f. said second inside diameter of said second hole is 0.8 to 1.2 times the first outside diameter;
g. said depth of the first hole ranges from equal to the thickness to 0.75 times said sum of the thickness and the length; and
h. said cut extends longitudinally from an end of the body farthest from the flange to the flange.

7. The device in claim 1 wherein;
a. said second outside diameter of said flange is 6 times said first outside diameter;
b. said third outside diameter of said body is 3 times the first outside diameter;
c. said thickness of the flange is 0.75 times the first outside diameter;
d. said length of the body is 5 times the first outside diameter;
e. said first inside diameter of said first hole is 2 times the first outside diameter;
f. said second inside diameter of said second hole is equal to the first outside diameter;
g. said depth of the first hole is 0.2 times said sum of the thickness and the length; and
h. said cut extends longitudinally from an end of the body farthest from the flange to the flange.

8. The device in claim 1 wherein said soft, pliable, and moldable elastomer comprises;
a. a silicone-based polymer suitable for non-implantable body contact and a Shore A hardness ranging from 20 to 80.

9. The device in claim 1 wherein said soft, pliable, and moldable elastomer comprises;
a. a polymer suitable for non-implantable body contact and a Shore A hardness ranging from 20 to 80.

10. A method to securely and dependably hold nasal prongs in a nasal cavity of a human by attaching a device made of a soft, pliable, and moldable elastomer to a cannula tube, said cannula tube having a first outside diameter, comprising;
a. selecting said device comprising;
(1) a flange comprising a first cylinder having a thickness and a second outside diameter;
(i) said thickness less than said second outside diameter;
(2) a body comprising a second solid cylinder having a length and a third outside diameter;
(i) said length greater than said third outside diameter;
(3) the second outside diameter is greater than the third outside diameter;
(4) said flange having a first longitudinal centerline;
(5) said body having a second longitudinal centerline;
(6) the flange indistinguishably and permanently joined to one end of the body so that said first longitudinal centerline and said second longitudinal centerline are coincident;
(7) a first hole passing through the flange with centerline equal to the first longitudinal centerline;
(i) said first hole having a first inside diameter less than the third outside diameter;
(8) a second hole passing through the body with centerline equal to the second longitudinal centerline;
(i) said second hole has a second inside diameter less than the third outside diameter and less than or equal to said first inside diameter;
(9) depth of the first hole as measured from top of the flange downward into the body is equal to or less than a sum of the thickness and the length; and
(10) a cut through side of the body;
b. attaching the device to the cannula tube so that the flange points towards the nasal prongs;
c. fitting the nasal prongs loosely in said nasal cavity;
d. stringing said cannula tube over each ear with the device loosely positioned behind said ear;
e. bringing the cannula tube to front of said human;
f. orientating the device so the flange faces said front of the human; and
g. positioning the device along the cannula tube.

11. The method in claim 10 wherein;
a. said second outside diameter of said flange is 4 to 12 times said first outside diameter;
b. said third outside diameter of said body is 2 to 6 times the first outside diameter;
c. said thickness of the flange is 0.2 to 4 times the first outside diameter;
d. said length of the body is 1 to 10 times the first outside diameter;
e. said first inside diameter of said first hole is 1.2 to 3 times the first outside diameter;
f. said second inside diameter of said second hole is 0.8 to 1.2 times the first outside diameter;
g. said depth of the first hole ranges from equal to the thickness to 0.75 times said sum of the thickness and the length; and
h. said cut courses spirally 180 to 720 degrees from an end of the body farthest from the flange to the flange.

12. The method in claim 10 wherein;
a. said second outside diameter of said flange is 6 times said first outside diameter;
b. said third outside diameter of said body is 3 times the first outside diameter;
c. said thickness of the flange is 0.75 times the first outside diameter;
d. said length of the body is 5 times the first outside diameter;
e. said first inside diameter of said first hole is 2 times the first outside diameter;
f. said second inside diameter of said second hole is equal to the first outside diameter;
g. said depth of the first hole is 0.2 times said sum of the thickness and the length; and
h. said cut courses spirally 360 degrees from an end of the body farthest from the flange to the flange.

13. The method in claim 10 wherein;
a. said second outside diameter of said flange is 4 to 12 times said first outside diameter;
b. said third outside diameter of said body is 2 to 6 times the first outside diameter;
c. said thickness of the flange is 0.2 to 4 times the first outside diameter;
d. said length of the body is 1 to 10 times the first outside diameter;
e. said first inside diameter of said first hole is 1.2 to 3 times the first outside diameter;
f. said second inside diameter of said second hole is 0.8 to 1.2 times the first outside diameter;
g. said depth of the first hole ranges from equal to the thickness to 0.75 times said sum of the thickness and the length; and
h. when viewed from the flange, said cut courses spirally clockwise or counterclockwise 90 to 270 degrees from an end of the body farthest from the flange to a longitudinal midpoint of the body and then spirally in an opposite direction 90 to 270 degrees from said longitudinal midpoint to the flange.

14. The method in claim 10 wherein;
a. said second outside diameter of said flange is 6 times said first outside diameter;
b. said third outside diameter of said body is 3 times the first outside diameter;
c. said thickness the flange is 0.75 times the first outside diameter;
d. said length of the body is 5 times the first outside diameter;
e. said first inside diameter of said first hole is 2 times the first outside diameter;
f. said second inside diameter of said second hole is equal to the first outside diameter;
g. said depth of the first hole is 0.2 times said sum of the thickness and the length; and
h. when viewed from the flange, said cut courses spirally clockwise or counterclockwise 180 degrees from an end of the body farthest from the flange to a longitudinal midpoint of the body and then spirally in an opposite direction 180 degrees from said longitudinal midpoint to the flange.

15. The method in claim 10 wherein;
a. said second outside diameter of said flange is 4 to 12 times said first outside diameter;
b. said third outside diameter of said body is 2 to 6 times the first outside diameter;
c. said thickness of the flange is 0.2 to 4 times the first outside diameter;
d. said length of the body is 1 to 10 times the first outside diameter;
e. said first inside diameter of said first hole is 1.2 to 3 times the first outside diameter;
f. said second inside diameter of said second hole is 0.8 to 1.2 times the first outside diameter;
g. said depth of the first hole ranges from equal to the thickness to 0.75 times said sum of the thickness and the length; and
h. said cut extends longitudinally from an end of the body farthest from the flange to the flange.

16. The method in claim 10 wherein;
a. said second outside diameter of said flange is 6 times said first outside diameter;
b. said third outside diameter of said body is 3 times the first outside diameter;
c. said thickness of the flange is 0.75 times the first outside diameter;
d. said length of the body is 5 times the first outside diameter;
e. said first inside diameter of said first hole is 2 times the first outside diameter;
f. said second inside diameter of said second hole is equal to the first outside diameter;
g. said depth of the first hole is 0.2 times said sum of the thickness and the length; and
h. said cut extends longitudinally from an end of the body farthest from the flange to the flange.

17. The method in claim 10 wherein said soft, pliable, and moldable elastomer comprises;
a. a silicone-based polymer suitable for non-implantable body contact and a Shore A hardness ranging from 20 to 80.

18. The method in claim 10 wherein said soft, pliable, and moldable elastomer comprises;
a. a polymer suitable for non-implantable body contact and a Shore A hardness ranging from 20 to 80.

19. A device made of a soft, pliable, and moldable elastomer to securely and dependably hold nasal prongs in a nasal cavity of a human by attachment to a cannula tube, said cannula tube having a first outside diameter, comprising;
a. a flange comprising a first cylinder having a thickness and a second outside diameter;
(1) said thickness is equal to said first outside diameter and said second outside diameter is 6 times the first outside diameter;
b. a body comprising a second solid cylinder having a length and a third outside diameter;
(1) said length is 5 times the first outside diameter and said third outside diameter is 3 times the first outside diameter;
e. said flange having a first longitudinal centerline;
f. said body having a second longitudinal centerline;
g. the flange indistinguishably and permanently joined to one end of the body so that said first longitudinal centerline and said second longitudinal centerline are coincident;
h. a first hole passing through the flange with centerline equal to the first longitudinal centerline;
(1) said first hole has a first inside diameter 1.5 times the first outside diameter;
i. a second hole passing through the body with centerline equal to the second longitudinal centerline;
(1) said second hole has a second inside diameter equal to the first outside diameter;
j. depth of the first hole as measured from top of the flange downward into the body is 0.2 times a sum of the thickness and the length;
k. a cut extending through side of the body;
(1) said cut courses spirally 360 degrees from an end of the body farthest from the flange to the flange; and
l. said soft, pliable, and moldable elastomer comprises;
(1) a silicone-based polymer suitable for non-implantable body contact and a Shore A hardness ranging from 20 to 80.
